(19) United States
(12) Patent Application Publication
(10) Pub. No.: US 2008/0311162 A1
(43) Pub. Date: Dec. 18, 2008

(54) Solid form

(76) Inventors: Olivia Darmuzey, Brussels (BE); Graeme MacLeod, Wezembeek Oppem (BE); Dzenana Cengic, Brussels (BE)

Correspondence Address: PATENT ADMINISTRATOR;FMC CORPORATION, 1735 MARKET STREET, PHILADELPHIA, PA, 19103, US

(21) Appl. No.: 11/803,825

(22) Filed: May 16, 2007

(51) Int. Cl. A61K 8/02; A61K 9/14; A61K 31/522; C11D 17/06

(52) U.S. Cl. 424/401; 424/490; 514/263.34

(57) ABSTRACT

A solid form comprising at least one film enrobing a compacted fill material comprising a pressure sensitive multiparticulate and at least one cushioning agent, in which the multiparticulate and/or the cushioning agent comprises at least one active material, having low friability and wherein the compacted fill material has a density of at least 0.5 g/ml based on the total solid volume of the solid form and a tensile strength of less than 0.9 MPa.

### FIELD OF THE INVENTION

The invention relates to a solid form comprising a film enrobing a compacted fill material, wherein the compacted fill material comprises a pressure sensitive multiparticulate and at least one cushioning agent and in which the multiparticulate and/or cushioning agent comprises an active material. The present invention is also directed to a method of making and using such a solid form.

### BACKGROUND TO THE INVENTION

Active ingredients, for example pharmaceutical, agrochemical and detergent active ingredients may be delivered through a wide range of solid forms including tablets and capsules. Conventional tablets generally are highly compacted and have relatively high densities. In conventional tablets, the active ingredient is generally compacted with other components in a blend to provide the requisite structural integrity for the tablet. Delivery of the active ingredient in use may however be unsatisfactory due to the compaction level and it is known to add excipients to the formulation to aid disintegration or dissolution of the tablet to improve delivery, aid compaction, increase strength and increase robustness of the solid form. This may however impose constraints on the flexibility of the formulator in developing tablets containing the active ingredient.

Capsules generally include the active ingredient in a relatively non-compacted form. However, the lack of compaction together with the void space inherent within capsules mean that for a given large dose of active, the volume of the final solid form is greater than for more compacted solid forms. Increasing the size of the capsule to accommodate the required dose is undesirable for the user. Typically, capsules require a relatively high level of disintegrant to provide adequate disintegration of the solid form. Capsule shells may also be sensitive to moisture and present problems as regards storage and product shelf-life.

WO 03/096963 discloses solid forms and processes utilizing films to enrobe a fill material to a degree of compaction less than that generally used to make a tablet. It is specifically disclosed therein that because of the nature of the capsule produced that certain ancillary ingredients necessary in conventional tablet production may be omitted. It is further disclosed therein that, due to relatively loose compaction, components contained within a tablet which are “designed to disperse and breakup the tablet when it has reached the site of delivery, can be omitted, as the active ingredients in the capsule according to the present invention are in a non-compacted or at least less compacted form as compared to a conventional tablet, and this lesser compaction leads to the easy release and dispersal of active ingredients once the capsule film has dissolved, e.g., at the intended site of delivery”.

There remains a need to provide a solid form able to deliver an active material, for example to provide a suitable dose level and a therapeutically beneficial delivery of the active material in a pharmaceutical product, and to retain flexibility for the formulator as to the components employed in the solid form. In making the solid form, application of sufficient pressure to the components in formation of the solid form to provide adequate structural integrity to the solid form may however be problematic due to the sensitivity to pressure of certain components. This sensitivity to pressure may result in the intended release profile of the active material being altered during production, distribution or storage prior to use.

### SUMMARY OF THE INVENTION

The present inventors have found that a solid form having a compacted fill material with a particular combination of components in which the components are less compacted than in a tablet but more than in a capsule formulation and which contains a pressure sensitive material in particulate form wherein each particle is composed of a plurality of smaller particles which are bound together, hereinafter referred to as a “multiparticulate”, and a cushioning agent and in which the multiparticulate and/or cushioning agent comprises an active material, ameliorate this problem. Sufficient pressure may e applied during the formation of the solid form to provide acceptable structural integrity without damaging the pressure sensitive component and adversely affecting the release profile of the active in the solid form.

The invention provides in a first aspect a solid form comprising at least one film enrobing a compacted fill material wherein:
i) the compacted fill material comprises
   a) a pressure sensitive multiparticulate; and
   b) at least one cushioning agent;
ii) the pressure sensitive multiparticulate and/or the cushioning agent comprises at least one active material;
iii) the solid form has a weight loss that is less than 1% during a 30 minute friability test in accordance with United States Pharmacopeia 29 Test Number 1216;
iv) the compacted fill material has a density of at least 0.5 g/ml based on the total solid volume of the solid form and a tensile strength of less than 0.9 MPa.

### DETAILED DESCRIPTION OF THE INVENTION

The term “multiparticulate” is known to those skilled in the art. As used herein, “multiparticulate has the meaning known to those killed in the art and refers to a material having discrete particles, each of which particle is itself composed of smaller particles which are bound together by physical or chemical interactions to produce the multiparticulate. Examples of multiparticulates include pellets, granules, spheres, microspheres, freeze dried material and crystals. The multiparticulate for use in the present invention may be coated or uncoated. Multiparticulates can have any shape and texture and can be produced by known processes. When taken orally, the multiparticulate suitably disperses freely in the gastrointestinal tract, optimizes absorption, and can minimize side effects. A multiparticulate may contain one or more components.

As used herein, the term “pressure sensitive multiparticulate” means a multiparticulate that has a physical attribute or characteristic for example its rate of dissolution, efficacy, or mechanical strength altered detrimentally to a material extent when the multiparticulate is compacted as compared to the uncompacted multiparticulate. Appropriate tests to determine whether an attribute or characteristic has been detrimentally affected as a result of compaction of the multiparticulate will depend on the particular characteristic being measured and are known to the skilled person.

As used herein, the term “modified release” refers to the solid form of the present invention having fast, pulsatile, delayed and/or controlled release characteristics as desired which have been modified as compared to an immediate release profile and are terms known in the art.

The term “immediate release” solid form as used herein refers to a solid form in which the active material is released rapidly after administration. A typical release rate for an “immediate release” solid form is suitably not less than 85% active material release in 60 minutes, preferably in 45 minutes and especially in 30 minutes in the test specified in the USP Edition 29 Test Number 711 at page 2673 for said active material when said active material is placed in a dissolution medium as specified in the USP dissolution specification or selected from dissolution media specified in the USP according to the solubility properties of said active material. This is referred to in the USP as “Q” time. The term “immediate release” includes “fast release”.

The solid form may comprise an active material which exhibits immediate release. The solid form may additionally comprise an active material which does not exhibit immediate release. If desired, the solid form may comprise an active material which exhibits immediate release and be free of an active material which does not exhibit immediate release.

Preferably, the at least one of the active material has a mean dissolution of at least 75% in 300 seconds in the test specified in the USP Edition 29 Test Number 711 at page 2673 for said active material when the active material is placed in a dissolution medium as specified in the USP dissolution specification or selected from dissolution media specified in the USP according to the solubility properties of the active material or as selected by the skilled person for example selected from: (i) the USP for the at least one active material, (ii) water, (iii) 0.1 M HCl or (iv) phosphate buffer having a pH between 5.8 and 8.0.

A solid form having an active material meeting this dissolution test is considered herein to be a “fast release” solid form. The solid form preferably comprises an active material exhibiting a fast release. The solid form may comprise a further active material which does not exhibit fast release. As desired, the solid form does not contain an active material which does not exhibit a fast release.

Suitable active materials include a pharmaceutical active, food component or product, veterinary active, cosmetic component or product, an appetite suppressant, detergent component or product or nutraceutical component or product. Preferably, the solid form comprises at least one film enrobing a compacted fill material wherein the compacted fill material comprises a pressure sensitive multiparticulate and at least one cushioning agent, the pressure sensitive multiparticulate and/or the cushioning agent comprising at least one active material, and the compacted fill material is selected from a pharmaceutical product, a food product, a veterinary product, a cosmetic, an appetite suppressant, a detergent product and a nutraceutical product, the said solid form shows a weight loss that is less than 1% during a 30 minutes USP friability test United States Pharmacopeia (USP) 29 Test Number 1216 (page 3046).

The active material preferably is present as a component of the pressure sensitive multi-particulate or of the cushioning agent or both. The active material may perform the function of a pressure sensitive agent or a cushioning agent as desired and the multiparticulate or cushioning agent may consist of the active material.

The film enrobing the compacted fill material is preferably a water-soluble film.

Desirably, the film is in intimate contact with the compacted fill material. By “intimate contact” it is meant that the film and the compacted fill material are in direct contact preferably over the entire internal surface of film although some areas not being in direct contact with the compacted fill material may be acceptable. For example, a tablet or other product form may be contained within the film or the film may have a lining or coating presenting a barrier between the compacted fill and the film.

The invention also provides a method of making a solid form comprising at least one film enrobing a compacted fill material, the solid form having a weight loss that is less than 1% during a 30 minute friability test in accordance with United States Pharmacopeia 29 Test Number 1216 and the compacted fill material having a density of at least 0.5 g/ml based on the total solid volume of the solid form and a tensile strength of less than 0.9 MPa, the said method comprising:
i) providing a first film shaped to define an interior volume for holding a compacted fill material and having an open end;
ii) depositing a fill material in the interior volume the fill material comprising a pressure sensitive multiparticulate and a cushioning agent and wherein at least one of the said multiparticulate and the cushioning agent comprises at least one active material;
iii) applying pressure to the fill material so as to compact the fill material;
iv) applying a second film over the said open end to close the said open end; and
v) sealing the first and second films together to enrobe the compacted fill material and provide the solid form.

In a further aspect, the invention provides a method of making a solid form comprising at least one film enrobing a compacted fill material, the solid form having a weight loss that is less than 1% during a 30 minute friability test in accordance with United States Pharmacopeia 29 Test Number 1216 and the compacted fill material having a density of at least 0.5 g/ml based on the total solid volume of the solid form and a tensile strength of less than 0.9 MPa, the said method comprising:
i) providing a first film shaped to define an interior volume for holding a compacted fill material and having an open end;
ii) depositing via the open end a first zone of a first fill material in the interior volume;
iii) depositing a second zone of a second fill material in the interior volume such that the interior volume comprises two zones of fill material wherein one of the first or second fill materials comprises a pressure sensitive multiparticulate and the other of the said first or second fill materials comprises a cushioning agent and wherein at least one of the said multiparticulate and the cushioning agent comprises at least one active material;
iv) applying pressure to the fill material so as to compact the at least two zones of fill material
v) applying a second film over the said open end to close the said open end; and
vii) sealing the first and second films together to enrobe the compacted fill material and provide the solid form.

Applicants have advantageously found that the solid form of the present invention comprising a pressure sensitive multiparticulate can be obtained at very low compression forces using at least one cushioning agent. Despite these relatively low forces the resultant solid form retains low friability and high integral strength. In addition, it has been shown that such a solid form protects the pressure sensitive multiparticulate which is being enrobed. This is generally achieved by the combination of the low forces applied and the use of at least one cushioning agent.

Advantageously, the solid form of the invention may be produced using low force and at least one cushioning agent, that in combination, protect the pressure sensitive multiparticulate from damage so as to avoid or reduce changes to the manner in which the active material is released in use from the solid form due to the manufacturing process. The manner of release of the active material in use is referred to herein and in the art as the “release profile” of the active material.

The compacted fill material preferably has a density of less than 1.1 g/ml and more preferably less than 1.05 g/ml. The density of the compacted fill material is suitably at least 0.55 g/ml, preferably, the density of the compacted fill material is from 0.55 to 1.04 g/ml, more preferably from 0.62 to 1.04 g/ml and desirably from 0.75 to 1 g/ml. The density of the solid form is suitably higher than that for conventional capsules and as the density contributes to the release profile of the solid form, this may be optimized by the formulator according to the release profile required.

The compacted fill material suitably has a tensile strength of less than 0.9 MPa, preferably less than 0.5 MPa, especially less than 0.2 MPa and particularly less than 0.1 MPa. The compacted fill material has sufficient tensile strength to retain the physical integrity of the compacted fill material and is preferably at least 0.05 MPa. The robustness of the solid form is suitably provided by the enrobing film rather than by the compacted fill material.

The solid form of the present invention has excellent robustness or physical strength. The robustness of a solid form may suitably be defined by measuring the weight loss of 10 solid forms when rotated in a USP friability apparatus. This test is as set out in USP 29<1216>p 3046. The solid form of the present invention shows a weight loss of less than 1% when tested for a 30 minutes in a friability drum. As conventional solid forms such as coated tablets are considered to be robust when the weight loss after 4 minutes of friability testing is less than 1% measured according to USP 29<1216>p 3046, the solid form of the present invention is especially robust.

The density of the compacted fill material of the solid form of the present invention refers to the total weight of the compacted fill material divided by the total volume of the solid form within the film material. This is typically referred to as the “apparent” density of the solid form. Unless otherwise stated or the context clearly requires, references to density herein are to “apparent” density.

The apparent density of a conventional tablet is typically greater than 1 g/ml as disclosed in, Pharmaceutical Technology, 27 (4), 67-80. In a conventional hard capsule, the fill material is lightly tamped so as to form a very weak slug that breaks up in the capsule shell, due to the air space above it. In a conventional hard capsule, the density of the fill material is therefore similar to the bulk density of the loose powder. The latter is typically less than 0.5 g/ml as disclosed in, Pharmaceutical Technology, 27 (4), 67-80. The density of the compacted fill material of the present invention is at least 0.5 g/ml based on the total solid form volume.

A typical method for determining the density D of the fill material in the present invention is to determine the fill weight W (1), the fill volume V, which depends on the size of the tooling used to manufacture the solid forms and to calculate D using equation (2).

(1) W=Wt−Wf (g), where Wt is the weight of the total enrobed solid form and Wf is the weight of the film enrobing the solid form.
(2) D=WN (g/ml)

For a solid form of the present invention having a 70 microns thick film and made with oblong concave tooling of 16.6 mm length and 7.3 mm width, the volume V of the fill material is calculated using equation (3)

(3) V=(212.7+110.8t)/1000 (ml), where t is the sidewall thickness of the solid form (mm), typically measured using a micrometer.

For a tablet or compact that is made using 13 mm diameter flat round punches, the volume V of the fill material is calculated using equation (4):

(4) V=[π(13/2)² t]/1000 (ml), where t is the tablet thickness (mm), typically measured using a micrometer.

Conventional tablets generally need to be robust for subsequent processing and handling such as film coating and packaging. Such tablets are considered to be robust when the tensile strength of the compacted material is at least 1.0 MPa for example as disclosed in Pharmaceutical Technology, p 52-62, April 2005 (Douglas McCormick,—Evolutions in Direct Compression).

A typical method for determining the tensile strength for round flat faced cylinder shapes is to measure the crushing force (also called hardness) of compacts on a tablet hardness tester and calculate the tensile strength a using equation (5) for example as disclosed in Journal of Pharmaceutical Sciences, vol. 59 (5), 688-691 “Determination of tablet strength by the diametral-compression test”, (Fell J. T. and Newton J. M., 1970),

(5) σ=2P/πDt (MPa), where P is the crushing force (N), D is the compact diameter (mm), and t is the compact thickness (mm), typically measured using a micrometer.

As used herein, the term “multiparticulate” refers to a particulate material, each particle of which is composed of agglomerated smaller particles which are bound together by physical or chemical interactions to produce a particle having a plurality of smaller particles bound together. The multiparticulate may for example be in the form of pellets, micronised powders, granules, spheres, microspheres, freeze dried material or crystals. The multiparticulate may be coated or uncoated. A multiparticulate as employed in this invention may have any shape and texture and can be produced by a range of processes.

When taken orally, a multiparticulate as employed in the present invention suitably disperses in the gastrointestinal tract and provides optimal absorption, and can minimize side effects. The multiparticulate may contain one or more components.

Suitably, at least one of said active material has a mean dissolution in the test specified in the USP Edition 29 Test Number 711 at page 2673 for said active material, which meets the USP dissolution specifications when said active material is placed in a dissolution medium as specified in the USP dissolution specification or selected from dissolution media specified in the USP according to the solubility properties of the active material. In an especially preferred embodiment, the at least one active material has a mean dissolution of at least 75% in 300 seconds when subjected to this dissolution test.

Where a dissolution medium is specified in the USP for an active material, this is suitably employed in the dissolution test. Where there is either:

i) no USP test for the active material;
ii) more than one test for the active material; or
iii) the active does not meet the USP specification with the specified medium;
the skilled person will select the most appropriate medium for the dissolution test from the USP dissolution media specified in the USP having regard to the dissolution characteristics of the active material.

Examples of media in which the dissolution test may be carried out include: (i) the medium specified in the USP preferably for said at least one active material, (ii) water, (iii) 0.1 M HCl or (iv) phosphate buffer having a pH between 5.8 and 8.0.

An active which has a mean dissolution of at least 75% in 300 seconds when subjected to this dissolution test set out above is considered to provide a “fast release” profile.

The term “cushioning agent” refers to a material, for example a cushion-like mass, capable of providing physical protection to the pressure sensitive multiparticulate by absorbing compaction stresses for example applied during manufacture or distribution such that attributes or characteristics of the multiparticulate are not adversely affected to a material extent.

In a preferred embodiment of the present invention, the solid form suitably contains at least two zones of compacted fill material. The zones may be of any shape or relative size and may have the same or different compositions but are suitably discrete. In a preferred embodiment, the zones are in the form of layers. Preferably, a first layer comprises the cushioning agent and a second layer comprises the pressure sensitive multiparticulate. The solid form may have multiple layers of cushioning agent and multiple layers of pressure sensitive multiparticulate. The relative location of each layer in the solid form may be selected as appropriate to the application.

In an especially preferred embodiment, a layer of pressure sensitive multiparticulate is disposed between two layers comprising a cushioning agent. Desirably, the multiparticulate is coated. Advantageously, the coated multiparticulate in combination with the low compaction level means that a disintegrant (to increase the disintegration rate of the solid form) need not be employed as part of the formulation as the multiparticulate fill, in use, disperses rapidly.

Another added benefit of the present invention is that the cushioning agent, for example, when present in top and bottom layers of the compacted fill material and positioned next to the film or in a physical blend with the multiparticulate, suitably gives to the solid form added smoothness and appearance.

In another embodiment of the present invention, the modified release solid form of the present invention contains a pressure sensitive multiparticulate in a physical blend with the cushioning agent. Preferably, the blend of the multiparticulate and the cushioning agent is substantially homogeneous.

The solid form may contain a mixture of the layered and blended embodiments of the invention as desired. For example, the solid form may comprise at least one layer of the cushioning agent and a second layer comprising a physical blend of pressure sensitive multiparticulate and cushioning agent.

Examples of suitable cushioning agents include, alone or in any combination acacia, alginic acid, calcium carbonate, calcium phosphate dibasic anhydrous, calcium phosphate dibasic dihydrate, calcium phosphate tribasic, carbomer, carboxymethylcellulose calcium, carboxymethylcellulose sodium, carrageenan, cellulose acetate, cellulose powdered, chitosan, citric acid, colloidal silicon dioxide, croscarmelose sodium, crospovidone, dextrates, dextrine, dextrose, dicalcium phosphate, ethylcellulose, fructose, gelatin, glucose, glyceryl behenate, glyceryl palmitostearate, guar gum, hydroxyethyl cellulose, hydroxyethylmethyl cellulose, hydroxypropyl cellulose, hydroxypropylmethyl cellulose, kaolin, lactitol, lactose, lactose calcium carbonate, low substituted hydroxypropyl cellulose, magnesium aluminum silicate, magnesium carbonate, magnesium oxide, maltodextrin, maltose, mannitol, methylcellulose, microcrystalline cellulose, pregelatinized starch, polacrilin potassium, polydextrose, polyethylene oxide, polymethacrylates, powdered cellulose, polyvinyl pyrrolidone, line cellulose, simethicone, sodium alginate, sodium bicarbonate, sodium chloride, sorbitol, sodium starch glycolate, starch, sucrose, sugar (e.g., compressible, confectioner's, spheres), talc, trehalose, xylitol, zein, crosslinked polyvinylpyrrolidone and low-substituted hydroxypropyl cellulose.

Where a layer of cushioning agent is employed, it is present in sufficient thickness to provide a cushioning effect during manufacture such that the release profile of the active is not materially altered. Preferably at least one portion of a first layer of cushioning agent has a thickness of at least 500 micrometers preferably at least 800 micrometers. If a second layer of cushioning agent is employed, then such second layer suitably has at least one portion thereof having a thickness of at least 500 micrometers and preferably at least 800 micrometers. Where two or more cushioning layers are employed, they are suitably of approximately the same thickness although they may be of different thickness if desired.

The pharmaceutical active can fall within any solubility class known in the art. It may include, for example, at least one pharmaceutical active that is a very soluble, freely soluble, soluble, poorly soluble or insoluble pharmaceutically active material. Suitably, the active material has a solubility in water of 1 g in less than 1 g water, 1 g in 1 to 10 g water, 1 g active in 10 to 30 g water, 1 g active in 30 to 100 g water, 1 g active in 100 to 1,000 g water, 1 g active in 1,000 to 10,000 g water, and 1 g active in more than 10,000 g water.

Examples of suitable classes of pharmaceutical actives include an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active, motion sickness active, anti-HIV, anti-malaria actives, anti-cholesterol actives, respiratory actives, gastronintestinal actives, cardiovascular actives, antidiabetes actives, central nervous system actives, anti-infection actives, mucolytics, proton pump inhibitors and nasal decongestants Examples of suitable actives include paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, pheneizine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocamide, timidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceutically or nutraceuticaly acceptable salts, acids, esters, isomers, and metabolites thereof.

Where more than one active material is present, the two or more actives may be from the same class or may be from different classes. Examples of combinations of active materials from different classes include an antibiotic in combination with one of a decongestant, an anti-inflammatory, a cough expectorant, a cough suppressant or an active for treatment of cough or common cold, a proton pump inhibitor, anti-hypertension and anti-cholesterol actives.

Examples of classes where two or more active materials from one class may suitably be employed include respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants, anti-HIV, anti-malaria actives.

Examples of particular combinations of active materials include: Paracetamol and Caffeine; Aspirin and paracetamol; Paracetamol and pseudoephedrine; Paracetamol and phenylephrine; Ibuprofen and codeine; Ibuprofen and pseudoephedrine; Paracetamol and diphenhydramine; Acravistine and pseudoephedrine; Paracetamol and dextromethorphan; Parcetamol and guaphenesin; Paracetamol, caffeine, aspirin; Aspirin and caffeine; Zidovudine, lamivudine and abacavir; Pravastatin and aspirin; Lamivudine and zidovudine; Roziglitazone and Metformin; Ezetimibe and fenofibrate; Amoxicillin and Clavulanate; Trimetoprim and sulfamethoxazole; Amlodipine and benazepril; Valsartan and Hydrochlorothiazide; Irbesartan and Hydrochlorothiazide; Losartan and Hydrochlorothiazide; Fenofibrate and Metformin; Abacavir and lamivudine; Dutasteride and tamsolusin; Atazanavir and ritonavir; Ritonavir and Saquinavir; Propoxyphene and paracetamol; Hydrocodone and paracetamol; tramadol and paracetamol; Metocarbamol and paracetamol; Memantine and Donepezil; Glyburide and Metformin; Pioglytazone and Metformin; Rosiglitazone and Glimepiride, Benazepril and Hydrochlorothiazide; Atorvastatin and Torcetrapib; Eprosartan and Hydrochlorothiazide; Amlodipine and Atorvastatin; Ezetimibe and Simvastatin; Telmisartan and Hydrochlorothiazide; Olmesartan and Hydrochlorothiazide; Lopinavir and Ritonavir; Emtricitabine and Tenofovir; Fosamprenavir and Ritonavir; Amprenavir and Ritonavir; Tipranavir and Ritonavir; Atovaquone and Proguanil; Lansoprazole, Amoxicillin and Clarithromycin; Lansoprazole and Naproxen; 5-aminosalicylic acid, 4-aminophthalic acid; Clarithromycin, Ranitidine and Bismuth citrate; Bismuth subsalicylate, Metronidazole and Tetracycline; Montelukast and Loratadine; Fexofenadine and pseudoephedrine; Guaifenesin and pseudoephedrine.

The pressure sensitive multiparticulate may be coated if desired. Any conventional coating may be employed for example a sustained release coating, enteric coating, taste-masking coating, moisture barrier coating, pressure sensitive barrier coating, pressure insensitive barrier coating and oxygen barrier coating and combinations thereof. Examples of such coating formulations suitably include at least one of methacrylates, methylcellulose, ethylcellulose, polyvinyl alcohol, hydroxypropylmethyl cellulose, hydroxypropyl cellulose, polyvinylacetate phthalate, methacrylic acid polymers, methacrylic ester copolymers, aminoalkyl methacrylate copolymers, hydroxypropylmethyl cellulose, carrageenan, ethylcellulose, starch acetates, polyethyl acrylate, polymethyl methacrylate, polymethacrylic acid, polyethyl acrylate, albumen, carboxymethyl cellulose, carboxymethylcellulose sodium, cellulose acetate, cellulose acetate phthalate, cetyl alcohol, chitosan, collagen, dextrin, gelatin, liquid glucose, glyceryl behenate, hyaluronic acid, hydroxyethyl cellulose, hydroxyethylmethyl cellulose, hydroxypropyl cellulose, hypromellose phthalate, lactose, maltitol, maltodextrin, methylcellulose, polydextrose, polyethylene oxide, polyvinyl acetate phthalate, polyvinyl alcohol, polyvinyl pyrilidone, sodium starch glycolate, shellac, carnauba wax, microcrystalline wax and zein, acethyltriethyl citrate, triethyl citrate, tributyl citrate, acetyltributyl citrate, dibutyl sebacate, diethyl phthalate, polyethylene glycol, 1,2-propylene glycol, glyceryl triacetate, glycerol, sorbitol, citric acid, lactic acid, triacetin, titanium dioxide, aluminium lakes, iron oxides, talc, magnesium stearate, glycerol monostearate, microcrystalline cellulose, colloidal silicon dioxide, precipitated silicon dioxide, magnesium Al silicate, crosslinked polyvinylpyrrolidone, starch, lactose, alginic acid, carboxymethylcellulose calcium, carboxymethylcellulose sodium, powdered cellulose, chitosan, croscarmellose sodium, crospovidone, guar gum, low-substitued hydroxypropyl cellulose, methylcellulose, polacrilin potassium, povidone, sodium alginate, sodium starch glycolate, starch, pregelatinized starch, simethicone emulsion, polysorbate, sodium carboxymethycellulose.

The film to be used to enrobe the present invention may be any film capable of enrobing the compacted fill materials without adversely impacting the desired dissolution profile. The film to be used may comprise water soluble components, water insoluble components or may comprise soluble and insoluble components in combination.

If desired, the film material may be designed to be fast, immediate, delayed, pulsatile or sustained release.

Preferably, the compacted fill material of the present invention is enrobed by a film comprising at least one water soluble polymer. Films generally useful in the present invention include those that are thermo formable and generally have a dissolution rate appropriate for the preparation of rapid release, preferably immediate release, solid forms of the invention. Examples of such water soluble polymers include cellulosic materials such as hydroxyethyl cellulose, hydroxypropyl cellulose, hydroxypropyl methyl cellulose; polyvinyl alcohol; hydrocolloids such as carrageenan, alginate and pectin; and water soluble acrylates. Examples of water insoluble polymers include ethylcellulose, methacrylates and cellulose acetate. The films used in the invention may be gelatin free. The films may contain plasticizers such as lactic acid, citric acid, polyethylene glycol, sorbitol, glycerine, triethylcitrate, propylene glycol, phthalates, triglycerides, triacetin, tributylcitrate, etc. WO 2004/026284, WO 02/083779 and WO 03/095548 disclose further examples of films that may be used in the invention and such are incorporated herein by reference. Examples of films that may be used in the present invention are available under the trade name XGEL UNO from BioTec Films LLC, Tampa, Fla., US. Films for use in the present invention may be made in a conventional manner. If desired, an adhesive and use thereof can be used to aid in sealing the films together. Suitable adhesive compositions include those set forth in WO 04/10337 and WO 04/103338—both of which are incorporated herein by reference.

The solid forms of the present invention may be enrobed and prepared in accordance with WO 03/096963, WO 05/030115, WO 05/030116 and PCT/GB2005/001077—all of which are incorporated herein by reference.

The solid form of the present invention may be prepared using any equipment capable of enrobing a compacted fill material for use in the present invention

The invention also provides for the use of a solid form according to the invention in which the active material comprises a pharmaceutical active in the manufacture of a medicament for treatment of the human or animal body by therapy.

A further aspect of the invention provides a solid form according to the invention in which the active material comprises a pharmaceutical active for use in a method of treatment of the human or animal body by therapy.

### DESCRIPTION OF THE DRAWINGS

In the Figures:

FIGS. 1 and 2 show microscope images of solid forms which have been coated. FIG. 1 shows pellets according to the invention and FIG. 2 shows damaged pellets presented for comparative purposes and are not according to the invention.

FIGS. 3 to 5 show the release profile of a pharmaceutical active from a solid form according to the invention.

The present invention is described by reference to the following illustrative examples. Unless otherwise indicated herein, all parts, percents, ratios and the like are by weight.

### EXAMPLES

The following materials, methods and equipment were used in the Examples set forth below unless otherwise indicated.

### Materials

TABLE 1

| Chemical name | Brand name | Manufacturer |
|---|---|---|
| Theophylline pellets (70% | | NATCO |
| active) | | |
| Poly (ethyl acrylate acid co- | Eudragit NE30D | Degussa |
| methyl methacrylate | | |
| Poly (ethyl acrylate-co- | Eudragit RS | Degussa |
| methyl methacrylate- | | |
| cotrimethylammonioethyl | | |
| methacrylate chloride) | | |
| Poly (ethyl acrylate-co- | Eudragit RL | Degussa |
| methyl methacrylate- | | |
| cotrimethylammonioethyl | | |
| methacrylate chloride) | | |
| Talc | | VWR |
| Triethyl citrate (TEC) | | MERCK |
| Silicon dioxide | Aerosil 200 pharma | Degussa |
| Cellulose microcrystalline | Avicel PH200 | FMC BioPolymer |
| microcrystlline cellulose, | LustreClear LC103 | FMC BioPolymer |
| iota carrageenan, | | |
| plasticizer | | |
| HPMC, | Enrobing film, 120 μm, | Biotech Film |
| Benzyl alcohol | | EM Science |
| hypromellose (HPMC) | Methocel ® E 15LV | Dow Chemical |
| Triacetin | | Eastman |
| Starch | Maize starch B | Roquette |
| Dicalcium Phosphate | | Astaris |
| anhydrous (DCP) | | |

### Equipment

Fluid bed; Glatt; Type: GPCG 3
Compaction simulator; ESH, oblong flat punches;
Solid form production apparatus as described in WO 05/030115; BioProgress;
Hardness tester; Erweka; TBH30MD
Dissolution bath; Hanson Virtual Instruments; SR8PLUS and SIP
Spectrophotometer; Hewlett Packard 8453
Microscope; SWIFT Instruments International; Stereo zoom eight
Balances (Mettler Toledo AG104, Mettler PM4800, Sartorius BP210S)
Mixers (Silverson L4R, IKA-WERKE)

### Methods

### 1) Coating Conditions:

Equipment: Fluid bed coater (Wurster technique)
(a) Preparation of Coating of Pressure Sensitive Multiparticulates with Eudragit NE30D:

TABLE 2
Eudragit NE30D suspension formulation

| Product | Percentage (%) | Weight (g) |
|---|---|---|
| Eudragit NE30D | 36.13 | 162.58 |
| Talc | 21.68 | 97.56 |
| Deionized Water | 42.19 | 189.86 |
| Total | 100.0 | 450.00 |

Talc was homogenized in water using a homogenizer (Silverson L4, high shear mixer) for 10 minutes. The prepared suspension was added slowly into the Eudragit NE30D dispersion while stirring gently with a conventional low shear mixer. Finally the suspension was passed through a 0.5 mm sieve. The suspension was continuously stirred during the application of the coating.

### (b) Preparation of Top Coating (Applied on Top of Eudragit Polymer Coating):

TABLE 3
LustreClear LC103 suspension formulation

| Product | Percentage (%) | Weight (g) |
|---|---|---|
| LustreClear LC103 | 90.00 | 40 |
| Deionized Water | 10.00 | 360 |
| Total | 100.00 | 400 |

The LustreClear LC103 was dispersed in water using propeller mixer (IKA-WERKE, low shear mixer) for 50 minutes.

### (c) Controlled Release Coating Method:

Coating with the Eudragit NE30D suspension was performed under the following conditions: Inlet temperature: 26° C.; Outlet temperature: 22° C.; Spray rate: 3.0-4.7 g/min; Air across bed: 0.30 kPa; Atomization pressure: 1.5 bar; Spraying time: 85 minutes. Targeted weight gain was 12%.
(d) Top Coating with LustreClear LC103 Method:
The coating with LustreClear LC103 was performed under the following conditions: Inlet temperature: 26° C.; Outlet temperature: 25° C.; Spray rate: 3.0 g/min; Air across bed: 0.35 kPa; Atomization pressure: 1.5 bar; Spraying time: 35 minutes; Drying time: 10 minutes in the fluid bed. Targeted weight gain was 1%.

### (e) Curing:

Prior to curing coated pellets were blended with 0.5% Aerosil 200. Pellets were cured for a curing time of 2 hours on a tray at an oven temperature of 45° C.

Note: coatings with Eudragit NE30D and LustreClear LC103 were both done on the same pellets in the same coater, one after the other. LustreClear LC103 is a second coating applied onto the Eudragit film. Its purpose is to decrease the sticking properties of the pellets coated with Eudragit NE30D.

(f) Preparation of Coating of Pressure Sensitive Multiparticulates with Eudragit RS/Eudragit RL Polymer:

TABLE 4
Eudragit RS/RL suspension formulation

| Product | Percentage (%) | Weight (g) |
|---|---|---|
| Eudragit RS | 32.3 | 242.5 |
| Eudragit RL | 3.6 | 27.0 |
| TEC | 2.2 | 16.5 |
| Talc | 5.4 | 40.5 |
| DI Water | 56.6 | 424.5 |
| Total | 100.0 | 750.0 |

The talc was homogenized in water using a homogenizer (Silverson L4, high shear mixer) for 10 minutes. The prepared suspension was added slowly into the Eudragit RS/RL dispersion while stirring gently with a conventional stirrer. Finally the suspension was passed through a 0.5 mm sieve. The suspension was continuously stirred during the coating process.

### (g) Preparation of Top Coating Applied on Top of Eudragit Polymer:

TABLE 5
LustreClear LC103 suspension formulation

| Product | Percentage (%) | Weight (g) |
|---|---|---|
| LustreClear LC103 | 90.00 | 20 |
| DI Water | 10.00 | 180 |
| Total | 100.00 | 200 |

The LustreClear LC103 was dispersed in water using propeller mixer (IKA-WERKE, low shear mixer) for 50 minutes.

### (h) Coating Method:

The coating with Eudragit NE30D suspension was performed under the following conditions: Inlet temperature: 37° C.; Outlet temperature: 27° C.; Spray rate: 2.0-3.8 g/min; Air across bed: 0.30 kPa; Atomization pressure: 1.5 bar; Spraying time: ˜180 minutes. Targeted weight gain was 10%.
(i) Top Coating with LustreClear LC103 Method
The coating with LustreClear LC103 was performed under the following conditions: Inlet temperature: 41° C.; Outlet temperature: 29° C.; Spray rate: 3.0 g/min; Air across bed: 0.35 kPa; Atomization pressure: 1.5 bar; Spraying time: ˜35 minutes; Drying time: 10 minutes. Targeted weight gain was 1%.

### (j) Curing:

Prior to curing coated pellets were blended with 0.5% Aerosil 200. Pellets were cured for a curing time of 24 hours on a tray at an oven temperature of 45° C.

Note: coatings with Eudragit RS/RL and LustreClear LC103 were both done on the same pellets in the same coater, one after the other. LustreClear LC 103 is second coating applied onto the Eudragit film. Its purpose is to decrease the sticking properties of the pellets coated with Eudragit RS/RL.

### 2) Fill Material Compacts Manufacture:

Fill material compacts were prepared by compressing the fill material in an ESH compaction simulator (ESH Testing Ltd, Dudley, UK) with oblong flat punches having a dimension of 16.775×7.449 millimeters. The upper punch speed was 100 mm/sec and the cycle time was 0.36 second. The upper punch displacement was adjusted to vary the compaction force. The maximum applied pressure was chosen for the pellets-cushioning agent blend in order to obtain solid forms with good tensile strength. In the case of tabletting of the pellet/cushioning agent blend the pressure applied was around 177 MPa. Low compaction examples used for the compacted fill material was undertaken at a pressure of around 4.4 MPa. This low compaction pressure was used for the compression of: pellets without cushioning agent, pellets with cushioning agent (pellets—cushioning agent blend and pellets—cushioning agent—pellets layers). The weight of solid forms produced at low compaction pressure was varied according to the individual experiment. It was typically in the range between 350 and 800 milligrams. Six compacts were prepared for dissolution testing. The upper punch force and compact weight were recorded. The compaction pressure reported was based on the upper punch force divided by the punch area (113.03 square millimeters). Compacts were stored in double plastic bags prior to subsequent testing.

Materials were filled into the compaction simulator dye manually. Two approaches to solid form manufacture were investigated: layering technique and blend dye loading.

### (a) Layering Technique:

A Layer of cushioning agent was loaded into the dye cavity of the compaction simulator or into the thermoformed cup of the enrobing Lab Scale Machine.
On the top of the cushioning agent layer a layer of the coated pellets was added.
Finally the coated pellets layer is covered again by a second layer of cushioning agent.

### (b) Blending Technique:

A measured amount of cushioning agent and coated pellets were loaded into the dye cavity. The blending was performed in the dye (manual blending).

### 3) Hardness Tests:

The hardness of the compacted dosages was evaluated on an Erweka hardness tester.

### 4) Microscope Observation:

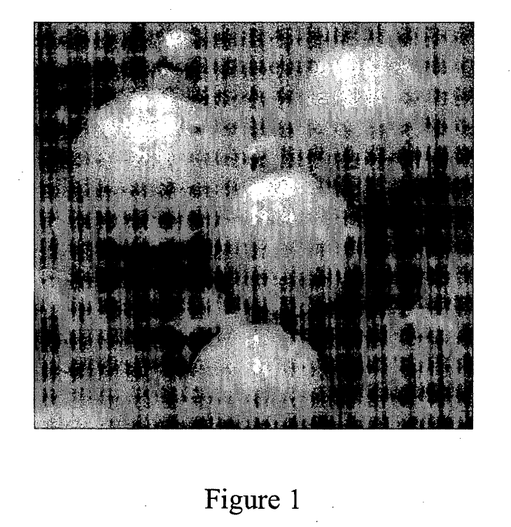

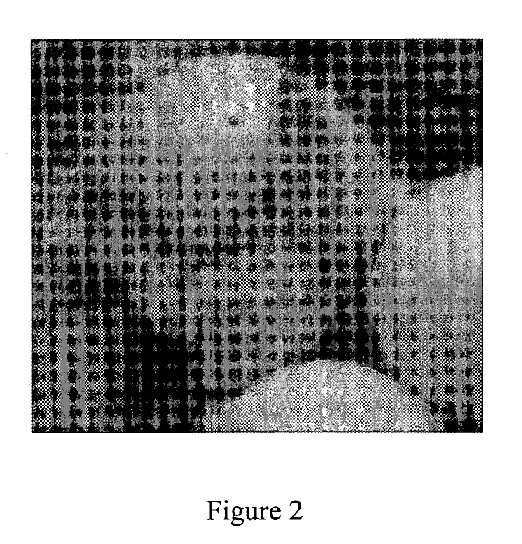

For easier observation through the microscope the dosages were gently dismantled (broken a part without causing any damage to the coated pellets). (FIGS. 1 and 2 show examples of undamaged and damaged pellets the latter being damaged during production due to the application of the compressive force or due to the absence of a cushioning agent. During the microscope observation the compacted dosages forms were thoroughly observed and any damage that occurred on the pellet coating monitored. Those pellets where the coating appeared damaged were counted (manual counting of damaged pellets).

The % damage to all pellets was calculated as follows:

$$X ( \% ) = \frac{\begin{matrix}[ number of damaged pellets \times \\ pellet weight ( mg ) ]\end{matrix}}{[ pellets layer weight ( mg ) ]} \times 100 ( \% )$$

Two-sample t-test: The two-sample t-test is used to determine if two population means are equal. A common application of this is to test if a new process or treatment is superior to a current process or treatment. (Montgomery D.C. (2001), Design and Analysis of Experiments, John Wiley & Sons, INC, 35-36, 640)

### 5) Manufacture of Enrobed SOLID Form:

Soluble films known as XGEL UNO and supplied by Bio Tec Films LLC were cut into strips 6 centimeters by 20 centimeters approximately. The lower and upper films had a thickness of about 120 microns. The lower film was heated sufficiently to thermoform under vacuum into dose cups about 3 millimeters in height to conform to cavities (7.5 millimeters width by 16.75 length millimeters) with the cavity depth determined by height-adjustable dose-shaped lower pistons within the stainless steel die. The film strip was placed over the die and brought in contact with a heated TEFLON® coated surface by means of upward vacuum. The film was then drawn into the stainless steel die cavities by inverting the vacuum to form a strip of twelve thermoformed dose cups with 3.0 millimeters separation between adjacent dose cups. Some unused portion of the filmstrip was cut and removed. The fill composition was dosed by volume using a Kinematics and controls dosing system. Solid forms were manufactured using the layering technique with three different fill layers: 1) 100 mg layer of Avicel PH200; 2) 150 mg layer of Theophylline pellets coated with Eudragit NE30D; 3) 100 mg layer of Avicel PH200.

The multiple doses were filled one on top of the others so as to form horizontal layers. Then the layered fill was lightly compacted in the dose cups with upper pistons, and the lower film was cut to separate the individual solid forms. The solid forms were then lifted by the lower pistons to expose a portion of the solid form sidewalls for application of the upper film to complete the enrobing of the solid form. An adhesive composed of 5% Methocel E15LV Premium, 45% Benzyl alcohol and 50% Triacetin, was applied (by transfer roller) to the upper filmstrip on the side to be pressed against the exterior sidewall of the dose cup. The upper film was placed over the solid forms containing the compressed layered powder fill and the film was heated by contact with the heating element using upper vacuum. The heated upper film was formed around the solid forms using the lower vacuum enclosing the fill material within the solid form by overlapping the upper film onto the sidewall of the solid forms. The top film was cut to separate the completed enrobed solid forms and the unused film was removed. The solid forms were further sealed by forcing them through a heated die under low pressure so that the cut film overlapping the sides was pressed smooth. All examples below used the apparatus set forth in WO 2005/030115,

Soluble HPMC containing films were used to enrobe the solid forms.

Dissolution was determined according to United States Pharmacopeia USP 24 with dissolution apparatus 1, baskets. The sampling was undertaken at the following intervals of time: time zero then at intervals of 30 min, 30 min, 1 hour, 2 hours and every 2 hours up to the end point of the given dissolution.

### Example 1

The theophylline pressure sensitive multiparticulates (pellets) were coated on the Glatt GPC with the Eudragit NE30D polymer, formulation shown in table 2. The batch size was 1 kg. The Eudragit NE30D polymer weight gain was 4%. The top coating (LustreClear LC103) was applied to the theophylline pellets coated with Eudragit NE30D. LustreClear weight gain 1%. Total theophylline pellets weight gain after both coating was 13%.

The coated pellets were submitted to either compaction or enrobing (see methods described above) and the resulting solid form attributes were compared to the non-compacted (free) pellets (Sample #1 in Table 6). Sample 5 in Table 6 is a Comparative Example due to its high density and Sample 6 is a Comparative Example due to the absence of a cushioning agent.

TABLE 6

| | | | | | | | Damaged | |
|---|---|---|---|---|---|---|---|---|
| | | | | CoP | CuA | Compaction | pellets | |
| | Solid | | Loading | quantity | quantity | pressure | coating | Hardness |
| # | form | Contents | method | (mg) | (mg) | (MPa) | (%) | (N) |
| 1 | Free | CoP | n/a | 400 | 0 | n/a | 0 | n/a |
| | pellets | | | | | | | |
| 2 | Solid | CuA, CoP | Layering | 150 | 2 × 100* | 4.4 | 3.0 | 120 |
| | form | | | | | | | |
| 3 | Compact | CuA, CoP | Layering | 150 | 2 × 100* | 4.4 | 3.5 | <10 |
| 4 | Compact | CuA, CoP | Blend | 150 | 200 | 4.4 | 3.5 | <10 |
| 5 | Compact | CuA, CoP | Blend | 150 | 200 | 176.9 | n/a** | 180 |
| 6 | Compact | CoP | n/a | 400 | 0 | 5.3 | >27 | <10 |

CuA: cushioning agent
CoP: coated pellets
*In the samples 2 and 3 the quantity of cushioning agent was composed of two layers each containing 100 mg of Avicel PH200
**In sample #5 the percentage of damaged pellets could not be evaluated because the compact was too hard and could not be dismantled into initial pressure sensitive multiparticulates without further damage of film coat

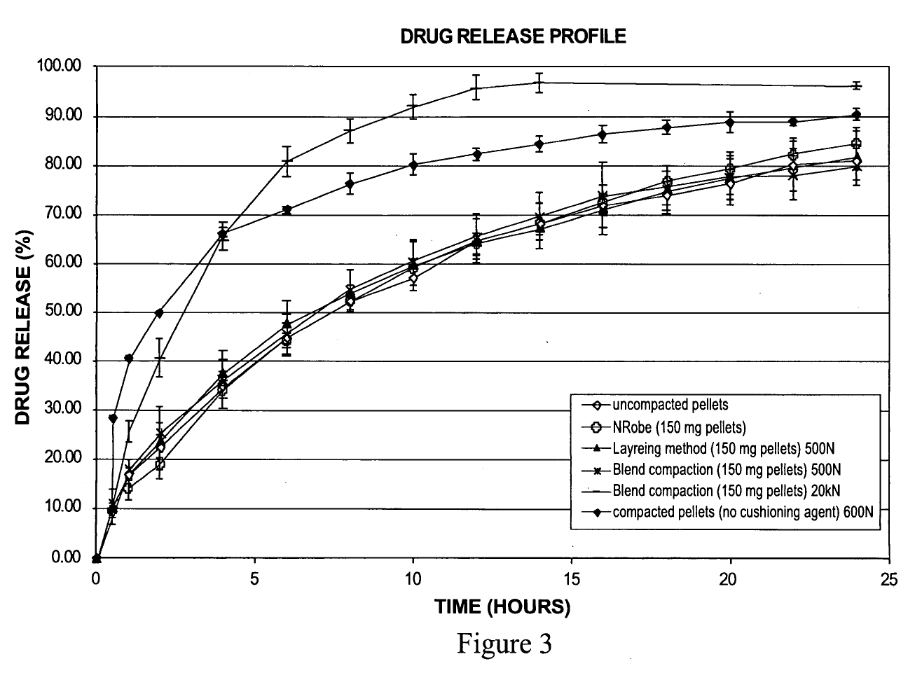

Samples 1 and 6 in Table 6 did not contain any cushioning agent. Sample 1 did not undergo any compression and served as a control sample. During the compression of Samples 3-6 the compaction simulator settings were adjusted in accordance with the pressures set forth in Table 6. The compacts produced at the low compaction pressure (4.4-5.6 MPa) had a very low hardness/tensile strength and in order to withstand the stress applied to the compact during post compaction handling a supporting enrobing film would be needed. Compaction pressures of 4.4 MPa would be expected to have a solid form density of lower than 1.1 g/ml, while a compaction pressure of 176.9 MPa (Sample 5) would be expected to provide an apparent density of greater than 1.1 g/ml. The drug release profiles for different samples set forth in Table 6 were evaluated as follows in Table 7 and illustrated in FIG. 3

TABLE 7

| Time (hours) | | | | | | | | | | | | | | |
|---|---|---|---|---|---|---|---|---|---|---|---|---|---|---|
| 0 | 0.5 | 1 | 2 | 4 | 6 | 8 | 10 | 12 | 14 | 16 | 18 | 20 | 22 | 24 |

Sample 1. Uncompacted pellets

| | | | | | | | | | | | | | | | |
|---|---|---|---|---|---|---|---|---|---|---|---|---|---|---|---|
| mean | 0.00 | 10.26 | 17.12 | 22.67 | 34.92 | 44.26 | 52.14 | 57.11 | 63.74 | 68.01 | 72.33 | 74.29 | 76.68 | 79.84 | 81.22 |

Sample 2. Enrobed solid form (150 mg pellets)

| | | | | | | | | | | | | | | | |
|---|---|---|---|---|---|---|---|---|---|---|---|---|---|---|---|
| mean | 0.00 | 9.60 | 14.55 | 19.27 | 34.15 | 44.41 | 52.72 | 59.10 | 64.18 | 68.36 | 72.07 | 76.70 | 79.34 | 82.07 | 84.38 |

Sample 3. Layering method (150 mg pellets) 4.4 MPa

| | | | | | | | | | | | | | | | |
|---|---|---|---|---|---|---|---|---|---|---|---|---|---|---|---|
| mean | 0.00 | 10.36 | 17.60 | 24.30 | 37.71 | 47.95 | 54.41 | 60.19 | 64.77 | 67.68 | 70.96 | 75.13 | 77.88 | 80.13 | 82.00 |

Sample 4. Blend compaction (150 mg pellets) 4.4 MPa

| | | | | | | | | | | | | | | | |
|---|---|---|---|---|---|---|---|---|---|---|---|---|---|---|---|
| mean | 0.00 | 11.15 | 17.95 | 25.16 | 36.41 | 45.47 | 54.60 | 60.40 | 65.51 | 69.69 | 73.99 | 75.29 | 76.92 | 78.25 | 79.97 |

Sample 5. Blend compaction (150 mg pellets) 176.9 MPa

| | | | | | | | | | | | | | | | |
|---|---|---|---|---|---|---|---|---|---|---|---|---|---|---|---|
| mean | 0.00 | 11.19 | 25.74 | 40.55 | 65.52 | 80.79 | 87.04 | 92.05 | 95.89 | 96.78 | X | X | X | X | 96.12 |

Sample 6. Compacted pellets (no cushioning agent) 5.3 MPa

| | | | | | | | | | | | | | | | |
|---|---|---|---|---|---|---|---|---|---|---|---|---|---|---|---|
| mean | 0.00 | 28.50 | 40.81 | 49.81 | 66.23 | 71.02 | 76.40 | 80.18 | 82.23 | 84.40 | 86.30 | 87.68 | 88.73 | 88.93 | 90.44 |

A two-sample T-test was undertaken on the samples in Table 6 to show the equivalency or non-equivalency, with statistical significance of the different drug release profile evaluated versus the drug release profile of the non compacted pellets (Sample 1). The t test was performed on the results obtained from the dissolution at 1, 4 and 12 hours. The t-test confidence limit is 95%. A t₀ value between: −2.228<t₀<2.228 shows equivalence.

T—test results for Sample 1 (Table 6) were compared to Sample 3 (Table 6) and are set forth in Table 8.

TABLE 8

| Sample evaluation time | |
|---|---|
| (hours) | t₀ value |
| 1 | −0.21 |
| 4 | −0.48 |
| 12 | 0.04 |

At all three measured times Sample 3 showed statistical equivalency to Sample 1 in its drug release profile.

T—test results for Sample 1 (Table 6) were compared to Sample 2 (Table 6) and are set forth in Table 9.

TABLE 9

| Sample evaluation time | |
|---|---|
| (hours) | t₀ value |
| 1 | 1.14 |
| 4 | 0.18 |
| 12 | −0.19 |

At all three measured times Sample 2 showed statistical equivalency to Sample 1 in its drug release profile.

T—test results for Sample 1 (Table 6) were compared to Sample 4 (Table 6) and are set forth in Table 10.

TABLE 10

| Sample evaluation time | T₀ |
|---|---|
| (hours) | value |
| 1 | −0.38 |
| 4 | −0.31 |
| 12 | −0.22 |

At all three measured times Sample 4 showed statistical equivalency to Sample 1 in its drug release profile.

T—test results for Sample 1 (Table 6) were compared to Sample 5 (Table 6) and are set forth in Table 11. In this case a t₀ value between: −2.365<t₀<2.365 show equivalence.

TABLE 11

| Sample evaluation time | |
|---|---|
| (hours) | t₀ value |
| 1 | −5.35 |
| 4 | −11.90 |
| 12 | −10.41 |

At all three measured times Sample 5 showed statistical non-equivalency to Sample 1 in its drug release profile.

T—test results for Sample 1 (Table 6) were compared to Sample 6 (Table 6) and are set forth in Table 12.

TABLE 12

| Sample evaluation time | |
|---|---|
| (hours) | t₀ value |
| 1 | −64.79 |
| 4 | −32.36 |
| 12 | −8.58 |

At all three measured times Sample 6 (having no cushioning agent and compacted at low pressure) showed statistical non-equivalency to Sample 1 in its drug release profile.

Table 13 summarizes the findings from Tables 8-12 when comparing Sample 1 (Table 6) to Samples 2-6 (Table 6).

TABLE 13

| Sample | Description | Release profile versus control |
|---|---|---|
| 2 | Layered solid forms of the | Equivalent |
| | present invention having low | |
| | fill density | |
| 3 | Layered Compacts having low | Equivalent |
| | apparent density | |
| 4 | Blended compacts having low | Equivalent |
| | apparent density | |
| 5 | Blended compacts having high | Not-equivalent |
| | apparent density | |
| 6 | Compacted pellets having low | Not-equivalent |
| | apparent density but without | |
| | cushioning agent | |

### Example 2

Coated pellets used in this Example 2 are from the same batch as the pellets coated in Example 1.

Samples 1-3 in Table 14 are the same samples as Samples 1-3, respectively, in Table 6 of Example 1. In this example the pellets in Sample 4 were compressed using the layering technique at the compaction pressure of 4.4 MPa and by varying the pellets layer quantity. These compacts consisted of a higher amount of coated pellets than the compacts in Samples 2-3.

TABLE 14

| | | | | | | | Damaged | |
|---|---|---|---|---|---|---|---|---|
| | | | | CoP | CuA | Compaction | pellets | |
| | | | Loading | quantity | quantity | pressure | coating | |
| # | Solid form | Contents | method | (mg) | (mg) | (MPa) | (%) | (N)** |
| 1 | Free | CoP | n/a | 400 | 0 | n/a | 0 | n/a |
| | pellets | | | | | | | |
| 2 | Enrobed | CuA, CoP | Layering | 150 | 2 × 100* | 4.4 | 3.0 | 120 |
| | solid form | | | | | | | |
| 3 | Compact | CuA, CoP | Layering | 150 | 2 × 100* | 4.4 | 3.5 | <10 |
| 4 | Compact | CuA, CoP | Layering | 250 | 2 × 100* | 4.4 | 4.5 | <10 |

CuA: cushioning agent
CoP: coated pellets
*In the samples 2, 3 and 4 the quantity of cushioning agent was composed of two layers each containing 100 mg of Avicel PH200
**Hardness

The coated pellets in Sample 1 (Table 14) did not undergo any compression and served as a control sample. Sample 2 (Table 14) was enrobed using the Enrobed solid form lab machine and Samples 3-4 (Table 14) were compressed using the compaction simulator. In samples 2-4 the cushioning agent was composed of two layers of Avicel PH200 (100 mg each). The amount of pellets in the layer varied from sample to sample. The compaction simulator settings were adjusted in order to obtain the compaction pressure of 4.4 MPa. Drug release profiles for different samples were evaluated using t-test analysis as set forth in Table 15. The data from Samples 1-3 in Table 14 is repeated from the same data in Table 7.

TABLE 15

| Time (hours) | | | | | | | | | | | | | | |
|---|---|---|---|---|---|---|---|---|---|---|---|---|---|---|
| 0 | 0.5 | 1 | 2 | 4 | 6 | 8 | 10 | 12 | 14 | 16 | 18 | 20 | 22 | 24 |

Sample 1 Uncompacted pellets from Table 6 and Table 14

| | | | | | | | | | | | | | | | |
|---|---|---|---|---|---|---|---|---|---|---|---|---|---|---|---|
| mean | 0 | 10.26 | 17.12 | 22.67 | 34.92 | 44.26 | 52.14 | 57.11 | 63.74 | 68.01 | 72.33 | 74.29 | 76.68 | 79.84 | 81.22 |

Sample 3 Layering method (150 mg pellets) 4.4 MPa from Table 6 and Table 14

| | | | | | | | | | | | | | | | |
|---|---|---|---|---|---|---|---|---|---|---|---|---|---|---|---|
| mean | 0 | 10.36 | 17.60 | 24.30 | 37.71 | 47.95 | 54.41 | 60.19 | 64.77 | 67.68 | 70.96 | 75.13 | 77.88 | 80.13 | 82.00 |

Sample 4 Layering method (250 mg pellets) 4.4 MPa from Table 14

| | | | | | | | | | | | | | | | |
|---|---|---|---|---|---|---|---|---|---|---|---|---|---|---|---|
| mean | 0 | 10.21 | 18.65 | 23.41 | 38.05 | 48.11 | 56.17 | 60.70 | 70.11 | 73.19 | 74.59 | 77.38 | 79.46 | 80.82 | 82.14 |

Sample 2 Enrobed solid form (150 mg pellets) from Table 6 and Table 14

| | | | | | | | | | | | | | | | |
|---|---|---|---|---|---|---|---|---|---|---|---|---|---|---|---|
| mean | 0 | 9.60 | 14.55 | 19.27 | 34.15 | 44.41 | 52.72 | 59.10 | 64.18 | 68.36 | 72.07 | 76.70 | 79.34 | 82.07 | 84.38 |

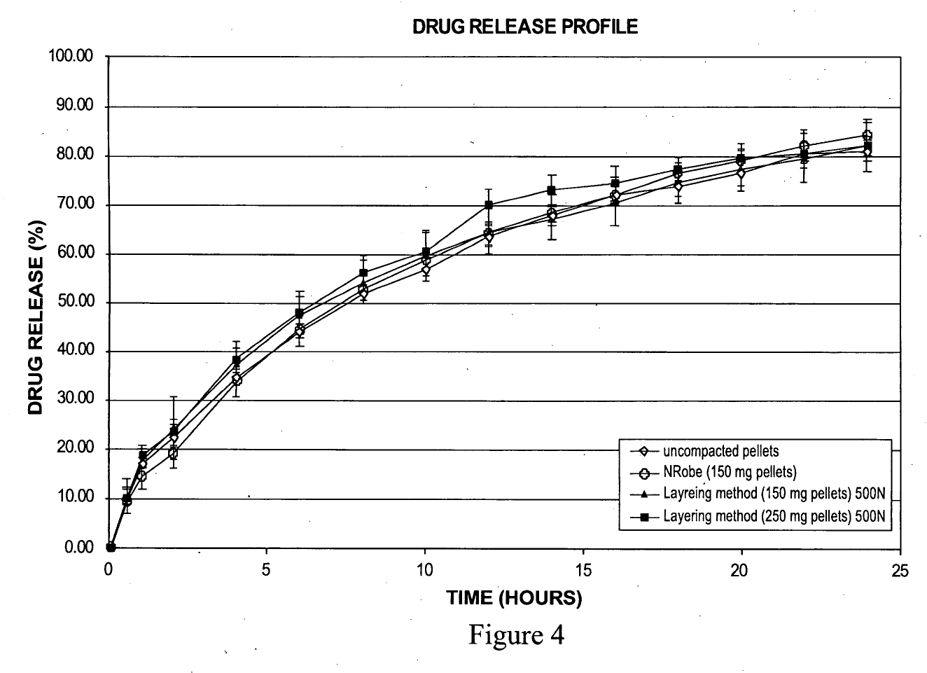

The obtained results are set forth in FIG. 4.

A two-sample T-test was undertaken on Sample 4 (Table 14) to show the equivalency or non-equivalency, with statistical significance of the different drug release profile evaluated versus the drug release profile of the non compacted pellets. The t test was performed on the results obtained from the dissolution at 1, 4 and 12 hours. The t-test confidence limit is 95%. A t₀ value between: −2.228<t₀<2.228 shows equivalence. The t test data for Samples 2 and 3 showed statistical equivalence as compared to Sample 1 as noted above.

T—test results for Sample 1 (Table 14) were compared to Sample 4 (Table 14) and are set forth in Table 16.

TABLE 16

| Sample evaluation time | |
|---|---|
| (hours) | t₀ value |
| 1 | −1.01 |
| 4 | −1.33 |
| 12 | −1.41 |

At all three measured times Sample 4 showed statistical equivalence to Sample 1 in its drug release profile. A summary is provided in Table 17.

TABLE 17

| Sample # | Description | Release profile versus control |
|---|---|---|
| 2 | Layered solid forms of the | Equivalent |
| | present invention | |
| 3 | Layered Compacts at low | Equivalent |
| | pressure 150 mg pellet layer | |
| 4 | Layered Compacts at low | Equivalent |
| | pressure 250 mg pellet layer | |
| | having a low apparent density | |

### Example 3

Coated pellets used in this Example 3 are from the same batch as the pellets coated in Example 1. In examples 1 and 2 the cushioning agent used was Avicel PH200. This example shows the effectiveness of different cushioning agents in the present invention. As the effect on release profile has been shown to be directly related to the degree of pellet coating damage, microscopic examination was undertaken (as it has previously been shown that this attribute is predictive of the drug release).

TABLE 18

| | | Cushioning | | Damaged |
|---|---|---|---|---|
| Sample | Cushioning | agent quantity | Pellets layer | pellets |
| # | agent | per layer (mg) | quantity (mg) | coating (%) |
| 1 | None (control) | 0 | 400 | >27 |
| 2 | Avicel PH200 | 100 | 150 | 4.0 |
| 3 | Starch | 155 | 150 | 1.9 |
| 4 | Dicalcium | 325 | 150 | 3.9 |
| | phosphate | | | |

In this example all dosages forms prepared and evaluated were compacts and the dosing method used was layering. Compaction was performed on Samples 1-4 in Table 18 using low pressure (4.4 MPa). During the compression the settings of the compaction simulator and the quantity of the pellets layer were kept unchanged. In order to obtain the compaction pressure of 4.4 MPa the quantity of the cushioning agent was adjusted accordingly. This adjustment in cushioning agent quantity was necessary because of different properties, e.g, density and consolidation of those three products evaluated. Table 18 shows the effectiveness of the inventive Samples 2-4 as compared to the degree of damaged pellets obtained for Sample 1.

### Example 4

Theophylline pellets (batch size of 1 kg), were coated with an Eudragit RS/Eudragit RL polymer blend (formulation methods described above in Table 4). The pellets polymer weight gain was 6.6%. Top coating was LustreClear LC103 applied onto the pellets coated with Eudragit RS/Eudragit RL. LustreClear weight gain was 1%. Total pellets weight gain after both coating was 11% w/w. In the following Table 19 the non compacted pellets (Sample 1) were compared to the pellets compacted with cushioning agent (Avicel PH200; Sample 2) using the layering technique and to the pellets compacted at 4.4 MPa without cushioning (Sample 3).

TABLE 19

| | | | | | | | Damaged |
|---|---|---|---|---|---|---|---|
| | | | | CoP | CuA | Compaction | pellets |
| | | | Loading | quantity | quantity | Pressure | coating |
| # | Solid form | Contents | method | (mg) | (mg) | (MPa) | (%) |
| 1 | Pellets | CoP | n/a | 400 | 0 | n/a | 0 |
| 2 | Compact | CoP, CuA | Layering | 150 | 2 × 100* | 4.4 | 4.0 |
| 3 | Compact | CoP | n/a | 420 | 0 | 4.4 | >25 |

CoP: coated pellets
CuA: cushioning agent
*In samples 2 the quantity of cushioning agent was composed of two layers each containing 100 mg of Avicel PH200

Sample 1 did not undergo any compression and served as a control sample. During the compression of sample 2 the compaction simulator settings were adjusted in order to obtain the desired compaction force.

TABLE 20
Drug release profiles of evaluated samples

| time (hours) | | | | | | | | | | |
|---|---|---|---|---|---|---|---|---|---|---|
| 0 | 0.5 | 1 | 2 | 4 | 6 | 8 | 10 | 12 | 14 | 16 |

Sample 1 Uncompacted pellets

| | | | | | | | | | | | |
|---|---|---|---|---|---|---|---|---|---|---|---|
| Mean | 0.00 | 0.32 | 0.83 | 1.60 | 14.43 | 28.24 | 42.89 | 57.37 | 71.50 | 82.36 | 93.92 |

Sample 2 Compacts 150 mg

| | | | | | | | | | | | |
|---|---|---|---|---|---|---|---|---|---|---|---|
| Mean | 0.00 | 3.32 | 3.06 | 4.09 | 16.38 | 28.91 | 41.79 | 53.30 | 64.91 | 76.22 | 86.29 |

Sample 3 Compacts No CuA

| | | | | | | | | | | | |
|---|---|---|---|---|---|---|---|---|---|---|---|
| Mean | 0.00 | 22.53 | 30.35 | 36.06 | 50.71 | 61.28 | 71.12 | 85.64 | 91.45 | 96.63 | 97.36 |

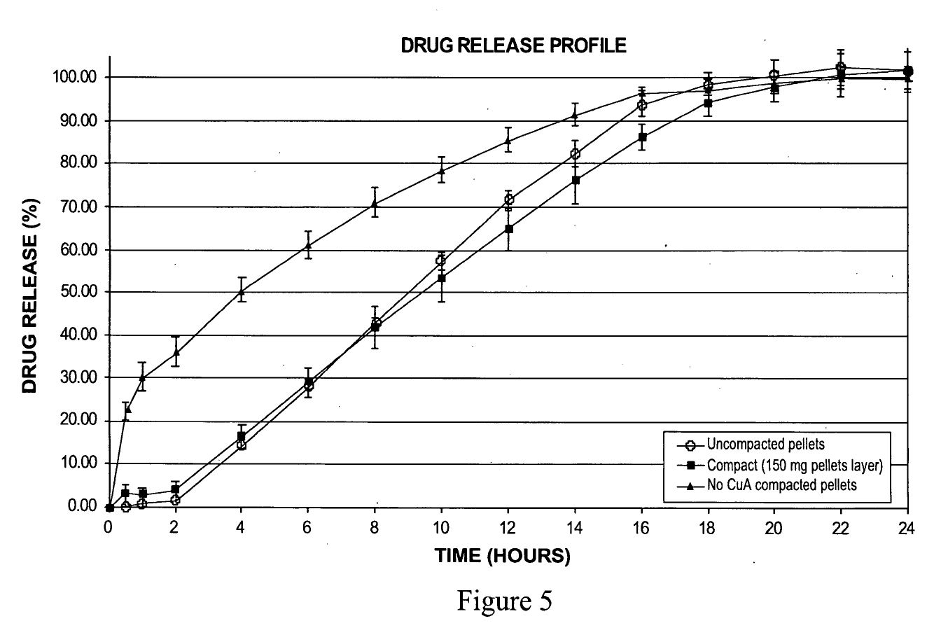

Obtained drug release profiles are set forth in FIG. 5.

A two-sample T-test was undertaken on Samples 1-3 to show the equivalency or non-equivalency, with statistical significance of the different drug release profile of Samples 2-3 evaluated versus the drug release profile of the non compacted pellets (Sample 1). The t test was performed on the results obtained from the dissolution at 4, 8 and 16 hours. In this case the first t-test was undertaken at the 4 hours time point because a significant lag time was observed in the release for the first 2 hours for all Samples 1-3 for this specific coating. Hence, a more significant data point is after the effects of this lag time have been taken into account.

T—test results for Sample 1 (Table 19) were compared to Sample 2 (Table 19) and are set forth in Table 21. In this case a t₀ value between: −2.080<t₀<2.080 shows equivalency.

TABLE 21

| Sample evaluation time | t₀ |
|---|---|
| (hours) | value |
| 4 | 1.12 |
| 8 | −0.20 |
| 16 | −1.99 |

At all three measured times Sample 2 showed statistical equivalency to Sample 1 in its drug release profile.

T—test results for Sample 1 (Table 19) were compared to Sample 3 (Table 19) and are set forth in Table 22. In this case a t₀ value between:: −2.120<t₀<2.120 shows equivalency.

TABLE 22

| Sample evaluation time | T₀ |
|---|---|
| (hours) | value |
| 4 | 26.42 |
| 8 | 13.03 |
| 16 | 0.81 |

Two samples out of three showed non-equivalency between the two drug release profiles evaluated for Sample 3 as compared to Sample 1.

While the invention has been described in detail and with reference to specific embodiments thereof, it will be apparent to one skilled in the art that various changes and modifications can be made therein without departing from the spirit and scope thereof.

1. A solid form comprising at least one film enrobing a compacted fill material wherein:
i) the compacted fill material comprises:
a) a pressure sensitive multiparticulate; and
b) at least one cushioning agent;
ii) the pressure sensitive multiparticulate and/or the cushioning agent comprises at least one active material;
iii) the solid form has a weight loss that is less than 1% during a 30 minute friability test in accordance with United States Pharmacopeia 29 Test Number 1216;
iv) the compacted fill material has a density of at least 0.5 g/ml based on the total solid volume of the solid form and a tensile strength of less than 0.9 MPa.

2. A solid form comprising at least one film enrobing a compacted fill material wherein the compacted fill material comprises a pressure sensitive multiparticulate and at least one cushioning agent, the pressure sensitive multiparticulate and/or the cushioning agent comprising at least one active material, and the compacted fill material is selected from a pharmaceutical product, a food product, a veterinary product, a cosmetic, an appetite suppressant, a detergent product and a nutraceutical product, the said solid form shows a weight loss that is less than 1% during a 30 minutes USP friability test United States Pharmacopeia (USP) 29 Test Number 1216 (page 3046).

3. A solid form according to claim 1 or claim 2 wherein said compacted fill material is present in two or more discrete zones.

4. A solid form according to claim 1 or claim 2 wherein the compacted fill material comprises multiple layers, wherein a first layer comprises said cushioning agent and a second layer comprises said pressure sensitive multiparticulate.

5. A solid form according to claim 4 wherein the compacted fill material further comprises a third layer comprising a cushioning agent the arrangement of the layers being such that the said pressure sensitive mulitparticulate layer is disposed between the two layers each comprising the said cushioning agent.

6. A solid form according to claim 1 or claim 2, wherein said compacted fill material comprises a physical blend comprising the pressure sensitive multiparticulate and the said cushioning agent.

7. A solid form according to claim 1 or claim 2, comprising at least one layer of said cushioning agent and a second layer comprising a physical blend of said pressure sensitive multiparticulate and said cushioning agent.

8. A solid form according to claim 1 or claim 2, wherein said at least one cushioning agent comprises at least one of acacia, alginic acid, calcium carbonate, calcium phosphate dibasic anhydrous, calcium phosphate dibasic dihydrate, calcium phosphate tribasic, carbomer, carboxymethylcellulose calcium, carboxymethylcellulose sodium, carrageenan, cellulose acetate, cellulose powder, chitosan, citric acid, colloidal silicon dioxide, croscarmelose sodium, crospovidone, dextrates, dextrine, dextrose, dicalcium phosphate, ethylcellulose, fructose, gelatin, glucose, glyceryl behenate, glyceryl palmitostearate, guar gum, hydroxyethyl cellulose, hydroxyethylmethyl cellulose, hydroxypropyl cellulose, hydroxypropylmethyl cellulose, kaolin, lactitol, lactose, lactose calcium carbonate, low substituted hydroxypropyl cellulose, magnesium aluminum silicate, magnesium carbonate, magnesium oxide, maltodextrin, maltose, mannitol, methylcellulose, microcrystalline cellulose, pregelatinized starch, polacrilin potassium, polydextrose, polyethylene oxide, polymethacrylates, polyvinyl pyrrolidone, line cellulose, simethicone, sodium alginate, sodium bicarbonate, sodium chloride, sorbitol, sodium starch glycolate, starch, sucrose, sugar, talc, trehalose, xylitol, zein, crosslinked polyvinylpyrrolidone and low-substituted hydroxypropyl cellulose.

9. A solid form according to claim 1 or claim 2, wherein said active material comprises at least one pharmaceutical active, nutraceutical active, food material, appetite suppressant, cosmetic component, detergent active, or industrial active material.

10. A solid form according to claim 9, wherein the said active material comprises at least one of a poorly soluble or insoluble pharmaceutically active material.

11. A solid form according to claim 9, wherein the said active material comprises at least one of a very soluble, freely soluble or soluble pharmaceutically active material.

12. A solid form according to claim 1 or claim 2, wherein said pressure sensitive multiparticulate comprises at least one multiparticulate that is a sustained release composition comprising said active material.

13. A solid form according to claim 1 or claim 2, wherein said pressure sensitive multiparticulate comprises at least one multiparticulate that is a delayed release composition comprising said active material.

14. A solid form according to claim 1 or claim 2, wherein said pressure sensitive multiparticulates comprise at least one multiparticulate that is a pulsatile release composition comprising said active material.

15. A solid form according to claim 1 or claim 2, wherein said pressure sensitive multiparticulate comprises at least one multiparticulate that is a fast release composition comprising said active material.

16. A solid form according to claim 1 or claim 2, wherein said pressure sensitive multiparticulate comprises at least one multiparticulate that is an immediate release composition comprising said active material.

17. A solid form according to claim 1 or claim 2, wherein said active material comprises at least one of an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active, motion sickness active, anti-HIV, anti-malaria actives, anti-cholesterol actives, respiratory actives, gastronintestinal actives, cardiovascular actives, antidiabetes actives, central nervous system actives, anti-infection actives, mucolytics, and nasal decongestants.

18. A solid form according to claim 1 or claim 2, comprising at least two active materials wherein the active materials are selected from:
i) an antibiotic in combination with a decongestant, an anti-inflammatory, a cough expectorant, a cough suppressant or an active for treatment of cough or common cold;
ii) an anti-HIV and anti-malaria active material;
iii) an anti-hypertension and anti-cholesterol active material and
iv) two or more active materials from the same class of active materials, the class being selected from respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants.

19. A solid form according to claim 1 or claim 2, wherein the said at least one active material comprises paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, PSE, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, pheneizine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocamide, timidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceuticaly or nutraceuticaly acceptable salts, acids, esters, isomers, and metabolites thereof.

20. A solid form according to claim 1 or claim 2, comprising at least two active materials wherein the active materials are selected from: Paracetamol and Caffeine; Aspirin and paracetamol; Paracetamol and pseudoephedrine; Paracetamol and phenylephrine; Ibuprofen and codeine; Ibuprofen and pseudoephedrine; Paracetamol and diphenhydramine; Acravistine and pseudoephedrine; Paracetamol and dextromethorphan; Parcetamol and guaphenesin; Paracetamol, caffeine, aspirin; Aspirin and caffeine; Zidovudine, lamivudine and abacavir; Pravastatin and aspirin; Lamivudine and zidovudine; Roziglitazone and Metformin; Ezetimibe and fenofibrate; Amoxicillin and Clavulanate; Trimetoprim and sulfamethoxazole; Amlodipine and benazepril; Valsartan and Hydrochlorothiazide; Irbesartan and Hydrochlorothiazide; Losartan and Hydrochlorothiazide; Fenofibrate and Metformin; Abacavir and lamivudine; Dutasteride and tamsolusin; Atazanavir and ritonavir; Ritonavir and Saquinavir; Propoxyphene and paracetamol; Hydrocodone and paracetamol; tramadol and paracetamol; Metocarbamol and paracetamol; Memantine and Donepezil; Glyburide and Metformin; Pioglytazone and Metformin; Rosiglitazone and Glimepiride, Benazepril and Hydrochlorothiazide; Atorvastatin and Torcetrapib; Eprosartan and Hydrochlorothiazide; Amlodipine and Atorvastatin; Ezetimibe and Simvastatin; Telmisartan and Hydrochlorothiazide; Olmesartan and Hydrochlorothiazide; Lopinavir and Ritonavir; Emtricitabine and Tenofovir; Fosamprenavir and Ritonavir; Amprenavir and Ritonavir; Tipranavir and Ritonavir; Atovaquone and Proguanil; Lansoprazole, Amoxicillin and Clarithromycin; Lansoprazole and Naproxen; 55-aminosalicylic acid, 4-aminophthalic acid; Clarithromycin, Ranitidine and Bismuth citrate; Bismuth subsalicylate, Metronidazole and Tetracycline; Montelukast and Loratadine; Fexofenadine and pseudoephedrine; Guaifenesin and pseudoephedrine.

21. A solid form according to claim 1 or claim 2, wherein said cushioning agent comprises a pharmaceutical active material.

22. A solid form according to claim 1 or claim 2, wherein said pressure sensitive multiparticulate comprises at least one of pellets, granules, spheres, microspheres, freeze dried material or crystals.

23. A solid form according to claim 1 or claim 2 wherein at least one of said pressure sensitive multiparticulates is coated and said coating comprises at least one of a sustained release coating, enteric coating, taste-masking coating, moisture barrier coating, pressure sensitive barrier coating, pressure insensitive barrier coating and oxygen barrier coating.

24. A solid form according to claim 23, wherein the coating comprises at least one of methacrylates, methylcellulose, ethylcellulose, polyvinyl alcohol, hydroxypropylmethyl cellulose, hydroxypropyl cellulose, polyvinylacetate phthalate, methacrylic acid polymers, methacrylic ester copolymers, aminoalkyl methacrylate copolymers, hydroxypropylmethyl cellulose, carrageenan, ethylcellulose, starch acetates, polyethyl acrylate, polymethyl methacrylate, polymethacrylic acid, polyethyl acrylate, albumen, carboxymethyl cellulose, carboxymethylcellulose sodium, cellulose acetate, cellulose acetate phthalate, cetyl alcohol, chitosan, collagen, dextrin, gelatin, liquid glucose, glyceryl behenate, hyaluronic acid, hydroxyethyl cellulose, hydroxyethylmethyl cellulose, hydroxypropyl cellulose, hypromellose phthalate, lactose, maltitol, maltodextrin, methylcellulose, polydextrose, polyethylene oxide, polyvinyl acetate phthalate, polyvinyl alcohol, polyvinyl pyrilidone, sodium starch glycolate, shellac, carnauba wax, microcrystalline wax and zein, acethyltriethyl citrate, triethyl citrate, tributyl citrate, acetyltributyl citrate, dibutyl sebacate, diethyl phthalate, polyethylene glycol, 1,2-propylene glycol, glyceryl triacetate, glycerol, sorbitol, citric acid, lactic acid, triacetin, titanium dioxide, aluminium lakes, iron oxides, talc, magnesium stearate, glycerol monostearate, microcrystalline cellulose, colloidal silicon dioxide, precipitated silicon dioxide, magnesium Al silicate, crosslinked polyvinylpyrrolidone, starch, lactose, alginic acid, carboxymethylcellulose calcium, carboxymethylcellulose sodium, powdered cellulose, chitosan, croscarmellose sodium, crospovidone, guar gum, low-substitued hydroxypropyl cellulose, methylcellulose, polacrilin potassium, povidone, sodium alginate, sodium starch glycolate, starch, pregelatinized starch, simethicone emulsion, polysorbate, sodium carboxymethycellulose.

25. A solid form according to claim 1 or claim 2, wherein said solid form further comprises a pressure-insensitive material.

26. A solid form according to claim 1 or claim 2 wherein the cushioning agent is present as a layer having a thickness of at least 500 micrometers.

27. A solid form according to claim 26, wherein at least one portion of the layer of cushioning agent has a thickness of at least 800 micrometers.

28. A solid form according to claim 1 or claim 2 comprising a first layer comprising a cushioning agent and a second layer comprising a pressure sensitive multiparticulate and a cushioning agent.

29. A solid form according to claim 1 or claim 2 wherein the tensile strength of the compacted fill material is less than 0.2 MPa.

30. A solid form according to claim 1 or claim 2 wherein said pressure sensitive multiparticulates are prepared by granulation, freeze drying, spray drying, roller compaction, lyophilization, extrusion, spheronization or milling.

31. A solid form according to claim 1 or claim 2, wherein the film enrobing the compacted fill material is a water-soluble film.

32. A solid form according to claim 1 or claim 2 in which the active material comprises a pharmaceutical active for use in a method of treatment of the human or animal body by therapy.

33. A method of treatment of the human or animal body by administering a solid form according to claim 1 or claim 2 in which the active material comprises a pharmaceutical, to the human or animal body.

34. A method of making a method of making a solid form comprising at least one film enrobing a compacted fill material, the solid form having a weight loss that is less than 1% during a 30 minute friability test in accordance with United States Pharmacopeia 29 Test Number 1216 and the compacted fill material having a density of at least 0.5 g/ml based on the total solid volume of the solid form and a tensile strength of less than 0.9 MPa, the said method comprising:
i) providing a first film shaped to define an interior volume for holding a compacted fill material and having an open end;
ii) depositing a fill material in the interior volume the fill material comprising a pressure sensitive multiparticulate and a cushioning agent and wherein at least one of the said multiparticulate and the cushioning agent comprises at least one active material;
iii) applying pressure to the fill material so as to compact the fill material;
iv) applying a second film over the said open end to close the said open end; and
v) sealing the first and second films together to enrobe the compacted fill material and provide the solid form.

35. A method of making a solid form comprising at least one film enrobing a compacted fill material, the solid form having a weight loss that is less than 1% during a 30 minute friability test in accordance with United States Pharmacopeia 29 Test Number 1216 and the compacted fill material having a density of at least 0.5 g/ml based on the total solid volume of the solid form and a tensile strength of less than 0.9 MPa, the said method comprising:
i) providing a first film shaped to define an interior volume for holding a compacted fill material and having an open end;
ii) depositing via the open end a first zone of a first fill material in the interior volume;
iii) depositing a second zone of a second fill material in the interior volume such that the interior volume comprises two zones of fill material wherein one of the first or second fill materials comprises a pressure sensitive multiparticulate and the other of the said first or second fill materials comprises a cushioning agent and wherein at least one of the said multiparticulate and the cushioning agent comprises at least one active material;
iv) applying pressure to the fill material so as to compact the at least two zones of fill material
v) applying a second film over the said open end to close the said open end; and
vii) sealing the first and second films together to enrobe the compacted fill material and provide the solid form.

36. A method of making a solid form according to claim 35 in which the first fill material comprises a cushioning agent, the second fill material comprises an optionally coated multiparticulate and which method further comprises depositing in a third zone a third fill material comprising a cushioning agent which may be the same or different to the first fill material.
